(19) United States
(12) Patent Application Publication
(10) Pub. No.: US 2025/0001158 A1
(43) Pub. Date: Jan. 2, 2025

(54) CARDIORENAL SYNDROME (CRS) THERAPY SYSTEMS AND METHODS

(71) Applicant: ABIOMED, Inc., Danvers, MA (US)

(72) Inventors: Michael Cuchiara, Danvers, MA (US); Soo Young Kim, Danvers, MA (US); Sathya Dev Unudurthi, Danvers, MA (US); Ralf Westenfeld, Danvers, MA (US)

(73) Assignee: ABIOMED, Inc., Danvers, MA (US)

(21) Appl. No.: 18/758,056

(22) Filed: Jun. 28, 2024

Related U.S. Application Data
(60) Provisional application No. 63/523,893, filed on Jun. 28, 2023.

(51) Int. Cl. A61M 60/33; A61M 60/135; A61M 60/237; A61M 60/414; A61M 60/515; A61M 60/585; A61M 60/894

(52) U.S. Cl. CPC A61M 60/33; A61M 60/135; A61M 60/237; A61M 60/414; A61M 60/515; A61M 60/585; A61M 60/894; A61M 2230/20

(57) ABSTRACT

A system and method for cardiorenal syndrome (CRS) therapy are provided. The system may utilize a therapy-delivery device configured to be disposed within a patient. The therapy-delivery device may be, e.g., a blood pump, a blood flow or pressure restrictor or enhancer. The system may include a controller operably coupled to the therapy-delivery device. The controller may be configured to adjust a therapy delivered by the therapy-delivery device based on a parameter related to renal function. The parameter may be measured by a real-time diagnostic sensor. The real-time diagnostic sensor may be integral to the therapy-delivery device, or may be located remotely from the therapy-delivery device, such as on a separate implantable device, or on a wearable medical device.

### CROSS-REFERENCE TO RELATED APPLICATIONS

The present application claims priority to U.S. Provisional Patent Application No. 63/523,893, filed 28 Jun. 2023, the contents of which are incorporated by reference herein in its entirety.

### TECHNICAL FIELD

The present disclosure is drawn to CRS therapies, such as techniques for selecting and/or modifying a therapy (e.g., via mechanical circulatory support devices) based at least in part on monitored parameters related to renal function.

### BACKGROUND

Cardiorenal syndrome (CRS) describes disorders involving both the heart and kidneys, in which acute or chronic dysfunction in one organ may induce acute or chronic dysfunction in the other organ. CRS represents heart-kidney interactions which in part are described by bidirectional hemodynamic crosstalk between the heart and the kidneys, as well as neurohormonal signaling.

### BRIEF SUMMARY

In various aspects, a system for cardiorenal syndrome (CRS) therapy may be provided. The system may include a therapy-delivery device configured to be disposed within a patient. The therapy-delivery device may be a blood pump, a blood flow or pressure restrictor or enhancer. The system may include a controller operably coupled to the therapy-delivery device. The controller may be configured to adjust a therapy delivered by the therapy-delivery device based on a parameter related to renal function.

The therapy-delivery device may be a blood pump disposed in a blood vessel forming part of a patient's arterial circulation or venous circulation. The therapy-delivery device may be a blood flow or pressure restrictor disposed in a blood vessel forming part of a patient's arterial circulation or venous circulation. The therapy-delivery device may be a blood flow or pressure enhancer disposed in a blood vessel forming part of a patient's arterial circulation or venous circulation. The parameter related to renal function may be a hemodynamic parameter, a renal function parameter, or a combination thereof. The hemodynamic parameter may be renal blood flow, cardiac index, or a combination thereof. The renal function parameter may be serum creatinine concentration, urine creatinine concentration, blood urea nitrogen (BUN), urinary output, net fluid output (fluid input-urine output), serum sodium concentration, urinary sodium concentration, total urinary sodium content, blood concentration or hematocrit, change in body weight, estimated glomerular filtration rate (eGFR), direct glomerular filtration rate (GFR), a renal injury marker, or a combination thereof. The renal injury marker may be Neutrophil gelatinase-associated lipocalin (NGAL) or Cystatin C.

The controller may be configured to receive data from a user (e.g., user-entered data), the data including the parameter related to renal function. The controller may be configured to receive the parameter related to renal function from a real-time diagnostic sensor. The real-time diagnostic sensor may be integrated into the therapy-delivery device. The real-time diagnostic sensor may be a separate device from the therapy-delivery device. The separate device may be an implantable device. The separate device may be a wearable device.

The real-time diagnostic sensor may be configured to detect an optical change in response to a measured biomarker. The real-time diagnostic sensor may be configured to detect a change in electrical properties in response to a measured biomarker. The real-time diagnostic sensor may be configured to detect a mechanical change in response to a measured biomarker. Adjusting the therapy may include increasing or decreasing therapy dose. Increasing or decreasing the therapy dose may include increasing or decreasing a rotational speed of a blood pump. Adjusting the therapy may include increasing or decreasing an intended duration of therapy. Adjusting the therapy may include increasing or decreasing a duty cycle of therapy. The controller may be configured to determine adequacy of therapy withdrawal timing and duration.

In various aspects, a method for cardiorenal syndrome (CRS) therapy may be provided. The method may include disposing a therapy-delivery device within a patient. The therapy-delivery device may be a blood pump, a blood flow or pressure restrictor or enhancer. The method may include receiving information related to renal function. The method may include adjusting a therapy delivered by the therapy-delivery device based on information related to renal function.

The therapy-delivery device may be a blood pump disposed in a blood vessel forming part of a patient's arterial circulation or venous circulation. The therapy-delivery device may be a blood flow or pressure restrictor disposed in a blood vessel forming part of a patient's arterial circulation or venous circulation. The therapy-delivery device may be a blood flow or pressure enhancer disposed in a blood vessel forming part of a patient's arterial circulation or venous circulation.

The parameter related to renal function may be a hemodynamic parameter, a renal function parameter, or a combination thereof. The hemodynamic parameter may be renal blood flow, cardiac index, or a combination thereof. The renal function parameter may be estimated glomerular filtration rate (eGFR), direct glomerular filtration rate (GFR), urinary output, urinary sodium concentration, total urinary sodium content, a renal injury marker, or a combination thereof. The renal injury marker may be Neutrophil gelatinase-associated lipocalin (NGAL), Cystatin C, TIMP-2 (Tissue Inhibitor of Metalloproteinase-2), IGFBP-7 (Insulin-like Growth Factor-Binding Protein 7), kidney injury molecule 1 (KIM 1), or a marker of renal oxygenation.

The method may include receiving information includes receiving data from a user. The method may include receiving information includes receiving data from a real-time diagnostic sensor. The method may include determining adequacy of therapy withdrawal timing and duration.

The real-time diagnostic sensor may be integrated into the therapy-delivery device. The real-time diagnostic sensor may be a separate device from the therapy-delivery device. The separate device may be an implantable device. The separate device may be a wearable device. The real-time diagnostic sensor may be configured to detect an optical change in response to a measured biomarker. The real-time diagnostic sensor may be configured to detect a change in electrical properties in response to a measured biomarker. The real-time diagnostic sensor may be configured to detect a mechanical change in response to a measured biomarker.

Adjusting the therapy may include increasing or decreasing therapy dose. Increasing or decreasing the therapy dose may include increasing or decreasing a rotational speed of a blood pump. Adjusting the therapy may include increasing or decreasing an intended duration of therapy. Adjusting the therapy may include increasing or decreasing a duty cycle of therapy. Adjusting the therapy may include the combination with other drug or device therapies. Other drug therapies may include those to tailor hemodynamics including vasoactive drugs (e.g., vasopressors, vasodilators, positive inotropic compounds) or may include those to provide renal protective effects (e.g., Cilastatin) or may include those that potentiate improved diuresis or natriuresis (e.g., diuretics), or a combination thereof.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1A is a block diagram of an embodiment of a system.

FIG. 1B is a block diagram of an alternate embodiment of a system.

FIGS. 2A, 2B, and 2C are illustrations of embodiments of a system.

FIG. 3 is a flowchart of an embodiment of a method for controlling a therapy-delivery device.

FIG. 4 is a flowchart of a method.

FIGS. 5 and 6 are flowcharts of alternative embodiments of methods for controlling a therapy-delivery device based on a parameter related to renal function.

FIG. 7 is a schematic illustration of a blood pump assembly.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

Cardiorenal syndrome (CRS) describes disorders involving both the heart and kidneys, in which acute or chronic dysfunction in one organ may induce acute or chronic dysfunction in the other organ. In some embodiments, CRS therapy delivery has a goal to protect the kidneys from damage and thereby stabilize renal function, improve renal function defined as increasing glomerular filtration rate (GFR), increasing diuresis and or natriuresis, without causing renal damage. As described herein, the inventors have appreciated the benefits of a system configured to tailor CRS therapy to measured renal function. In some embodiments, the disclosed therapy may include therapy via a mechanical circulatory support device.

To provide an overall understanding of the systems, method, and devices described herein, certain illustrative embodiments will be described. Although some embodiments and features described herein are specifically described for use with a mechanical circulatory device, e.g., an intracardiac heart pump system, it will be understood that all the components and other features outlined below may be combined with one another in any suitable manner, and may be adapted and applied to other types of medical devices such as electrophysiology study and catheter ablation devices, neuromodulation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

In some embodiments, CRS therapy delivery may involve increasing blood flow and or pressure into the renal artery and or increasing blood flow out of the renal vein and decreasing renal vein congestion (pressure). Taken together cardiorenal therapy may involve increasing the trans-renal pressure gradient (renal artery pressure-renal vein pressure).

In some embodiments, real-time/timely diagnostic information about renal function may inform cardiorenal therapy delivery by choosing the appropriate device which may be used for therapy (e.g., a venous device to reduce renal vein pressure, an arterial device to increase artery pressure, or both) guiding therapy initiation, therapy dose, therapy duration, and therapy weaning. The inventors have appreciated that integrating information about renal function with CRS therapy delivery may hold the promise to better deliver cardiorenal therapy.

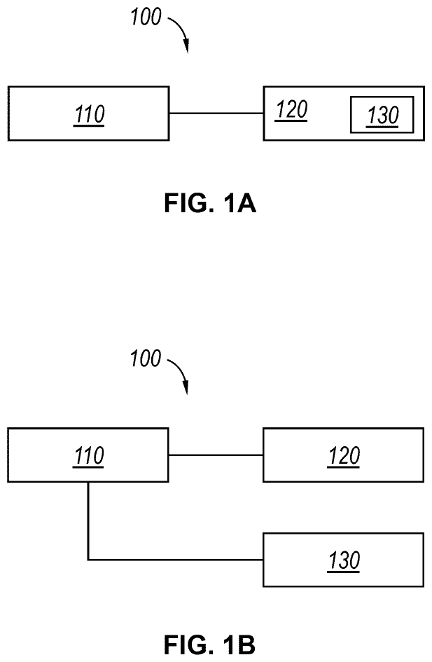

In some embodiments, a system for cardiorenal syndrome (CRS) therapy may be provided. Referring to FIG. 1A, a system (100) may include a therapy-delivery device (120) configured to be disposed within a patient. The therapy-delivery device may be a mechanical circulatory support device, such as a blood pump. The therapy-delivery device also may be a blood flow or pressure restrictor. In other embodiments, the therapy-delivery device may be a blood flow or pressure enhancer.

The therapy-delivery device may be a blood pump disposed in a blood vessel forming part of a patient's arterial circulation. The therapy-delivery device may be a blood pump disposed in a blood vessel forming part of a patient's venous circulation. The therapy-delivery device may be a blood flow or pressure restrictor disposed in a blood vessel forming part of a patient's arterial circulation. The therapy-delivery device may be a blood flow or pressure restrictor disposed in a blood vessel forming part of a patient's venous circulation. The therapy-delivery device may be a blood flow or pressure enhancer disposed in a blood vessel forming part of a patient's arterial circulation. The therapy-delivery device may be a blood flow or pressure enhancer disposed in a blood vessel forming part of a patient's venous circulation. In some embodiments, the therapy-delivery device may include a combination of these disclosed devices. In some embodiments, only a single therapy-delivery device may be provided. In some embodiments, a plurality of therapy-delivery devices may be provided.

In some embodiments, when a plurality of devices is provided, the devices may be disposed at same part of the patient's circulation (e.g., in arterial or venous circulation). For example, a blood pump and an inflatable balloon blood flow restriction device may both be located in a patient's venous circulation.

In some embodiments, when a plurality of devices is provided, the devices may be disposed at different locations. For example, in some embodiments, one device may be disposed in a patient's arterial circulation, and one device may be disposed in a patient's venous circulation.

The system may include a controller (110) operably coupled to the therapy-delivery device (120) (or to a plurality of devices). The controller may be configured to adjust a therapy delivered by the therapy-delivery device based on a parameter related to renal function. As described herein, the parameter related to renal function may be sensed in various ways. As shown in FIG. 1A, in some embodiments, a diagnostic sensor (130) may be integrated into the therapy-delivery device, where the diagnostic sensor is configured to provide the parameter. In some embodiments, the diagnostic sensor is a real-time diagnostic sensor. The diagnostic sensor also may be configured to measure the parameter at prescribed times (e.g., every 5 second, 10 seconds, 1 minute, 2 minutes, 5 minutes, 10 minutes, or longer), such as to provide feedback contemporaneous with a patient therapy.

Referring to FIG. 1B, the diagnostic sensor (130) may also be located remotely from the therapy-delivery device (120). For example, the diagnostic sensor may be disposed on an implantable device, such as a separate implantable medical device. The separate implantable medical device may be a second therapy-delivery device in some embodiments. The sensor or detector may be located extracorporeally. For example, the sensor or detector may be disposed on a wearable device. The sensor or detector also may be disposed remotely from the patient. For example, in some embodiments, the sensor or detector may be disposed on a cart holding the controller.

In some embodiments, the diagnostic sensor may be configured to detect an optical change in response to a measured biomarker. For example, the sensor may be configured to either directly or indirectly provide a response to the presence of a fluorescent taggant coupled to a biomarker that had previously been introduced to the bloodstream, that the kidney may then filter out over time. By understanding the rate of filtering, a condition of the kidney can be assessed.

The real-time diagnostic sensor may be configured to detect a change in electrical properties in response to a measured biomarker. For example, the sensor may be configured to detect a change in conductivity or resistance of the blood as the presence of the biomarker changes. The real-time diagnostic sensor may be configured to detect a mechanical change (e.g., a pressure, a flow rate, a force, etc.) responsive to a measured biomarker.

The parameter related to renal function may be a hemodynamic parameter, a renal function parameter, or a combination thereof. For example, a governing equation of direct glomerular filtration rate (GFR) may be GFR=RBF×filtration fraction, where RBF is a renal blood flow. It will be understood that data on its components may therefore help to guide therapy. The hemodynamic parameter may be renal blood flow, cardiac index, or a combination thereof. The renal function parameter may be estimated glomerular filtration rate (eGFR), which may be measured by, e.g., serum creatine. The renal function parameter may be direct glomerular filtration rate (GFR). The renal function parameter may be blood urea nitrogen (BUN). The renal function parameter maybe serum creatinine. The renal function parameter may be urinary output. The renal function parameter may be urinary sodium concentration. The renal function parameter may be total urinary sodium content. The renal function parameter maybe fractional sodium excretion (FeNa). The renal function parameter may be a renal injury marker. The renal injury marker may be Neutrophil gelatinase-associated lipocalin (NGAL), NAG (N-acetyl-beta-D-glucosaminidase), Cystatin C, TIMP-2 (Tissue Inhibitor of Metalloproteinase-2), IGFBP-7 (Insulin-like Growth Factor-Binding Protein 7), kidney injury molecule 1 (KIM 1), or a marker of renal oxygenation. The renal function parameter may be a combination of eGFR, GFR, BUN, SCr, urinary output, urinary sodium concentration, total urinary sodium content, and/or a renal injury marker.

There may be one or more thresholds for each renal function parameter to help guide therapy. For example, as is known, considering the NGAL marker, a range of <50 ng/ml indicates a low risk of acute kidney injury (AKI), a range of 50-149 ng/ml indicates an equivocal risk of AKI, a range of 150-300 ng/mL indicates a moderate risk of AKI, and a range of >300 ng/ml indicates a high risk of AKI. Similarly, the normal range of Cystatin C is around 0.62-1.15 mg/L, so values outside that range may indicate particular therapies are needed. Further, as is known, according to the optimal prediction of AKI, a [TIMP-2]. [IGFBP7] value of >0.3 (ng/ml) 2/1000) may be considered positive and a value of ≤0.3 (ng/ml) 2/1000 was considered negative. Alternatively, or in addition, in urine, KIM-1 concentration has been shown to increase up to 3-7 ng/ml from its normal concentration of less than 1 ng/ml; KIM-1 levels may begin to increase as early as, e.g., 6 hours after an ischemic insult and remained elevated for a period of 48 hours post-injury.

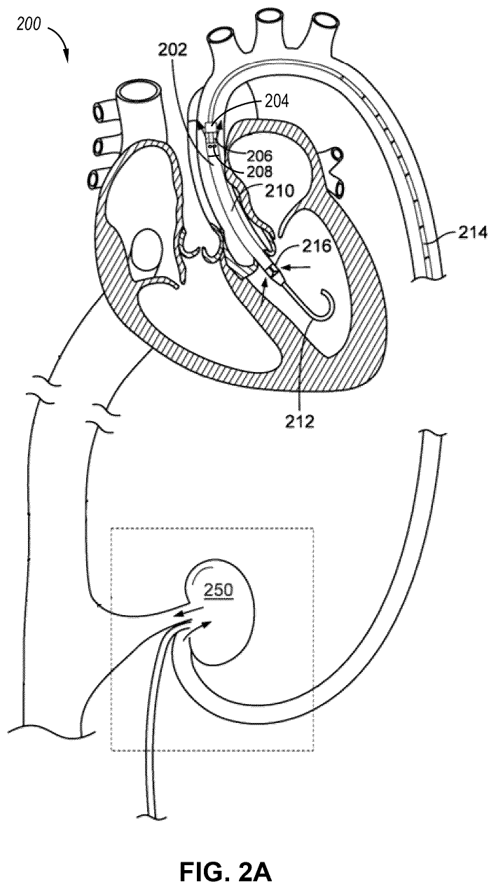

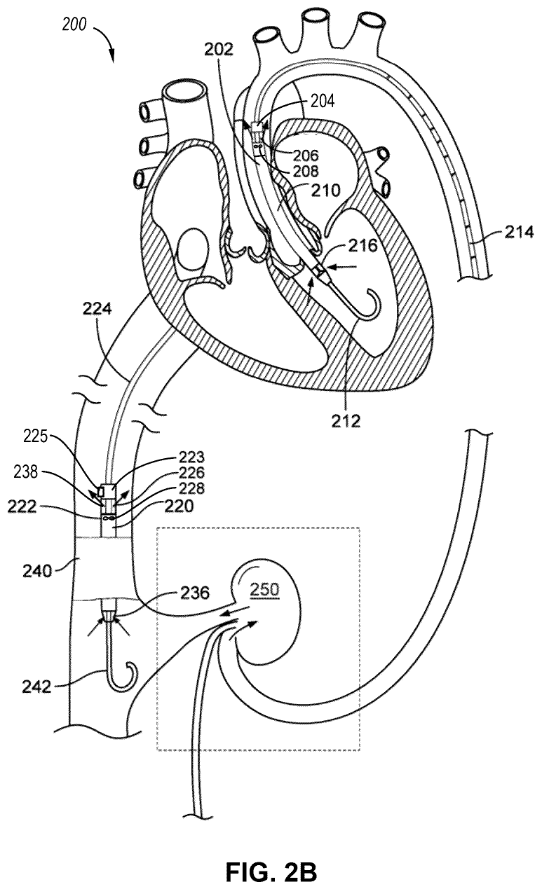

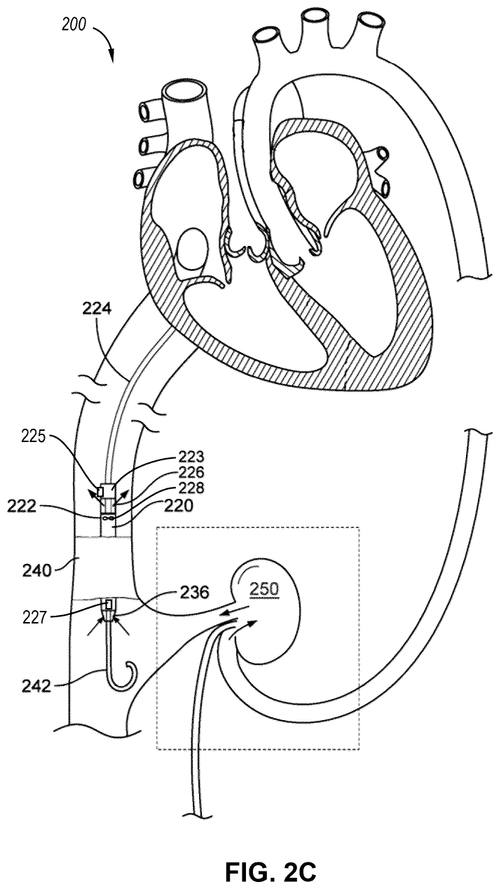

As an example, referring to FIGS. 2A-2C, embodiments of system (200) as shown. In some embodiments, as shown in FIG. 2A, the system may include a mechanical circulatory support device, such as pump (202) disposed in the patient's left ventricle. In some embodiments, as shown in FIG. 2B, the system may include first and second support devices, such as pump (202) and pump (222), disposed in the patient's inferior vena cava. In one such example, pump (202) and pump (222) may be IMPELLA® pumps from Abiomed, Inc. In other embodiments, the support devices may include other mechanical circulatory assist devices, such as an expandable pump, intra-aortic balloon pump or an extracorporeal membrane oxygenation system (ECMO).

In still other embodiments, such as those seen in FIG. 2C, the system may include only pump (222), which is disposed in the inferior vena cava.

In some embodiments pump (202) and/or pump (222) may control input and output into an organ, e.g., the heart and/or the kidney (250) (see FIGS. 2A and 2B). As will be appreciated, the kidney (250) may be the left kidney or the right kidney. In one example, pump (202) and/or pump (222) may control input and output into both kidneys. For example, operating one or both blood pumps may change a flow quantity and flow rate of blood through the organ, e.g., kidney. For example, operating one or both blood pumps may increase a flow quantity and flow rate of blood through the kidney.

As shown in FIG. 2A for example, pump (202) may be a first blood pump with motor (204) and rotor (206) in housing (208), cannula (210), distal extension (212), and catheter (214). As shown in the example of FIG. 2A, pump (202) may be placed with distal extension (212) in the left ventricle, and rotor (206) and housing (208) in the aorta. When operating, pump (202) may draw blood through the inlet (216), through cannula (210) and out through housing (208) (sometimes referred to as the rotor shroud), unloading the heart. This, in turn, may impact the functionality of the kidneys, as disclosed herein. In some embodiments, the distal extension (212) may act to stabilize pump (202) in the ventricle. For example, distal extension (212) may be a pigtail or j-shape. When operating, pump (202) may unload the left ventricle and increases pressure in the aorta, thereby increasing arterial pressure downstream (e.g., in the kidneys). Pump (202) may operate at a range of speeds resulting in a range of flow rates and associated increases in aortic pressure. For example, the pump (202) may be operated at a flow rate between about 1.5 L/min and 6 L/min. In one example, the pump (202) may be operated at a flow rate of about 5 L/min. Pump (202) may be percutaneously or surgically inserted into a patient via the femoral artery, or via the axillary artery or via the carotid artery.

As shown in FIG. 2B, pump (222) may be a second blood pump with motor (223) and rotor (228) in housing (226), cannula (220), distal extension (242), and catheter (224). As shown in the example of FIG. 2B, pump (222) may be placed in the inferior vena cava. When operating, pump (222) may draw blood through the inlet (236), through cannula (220) and out through housing (226). Housing (226) with rotor (228) may be in the inferior vena cava, downstream of inlet (236), also in the inferior vena cava. As will be understood by those of skill in the art, FIGS. 2A and 2B do not show all variations of the devices. For example, in some embodiments, the housing (226) with rotor (228) in pump (222) may be upstream of the cannula (220), such that blood is pushed through the cannula, exiting at the outlet (238). A similar design may be used for pump (202).

In one example, e.g., as shown in FIG. 2, pump (222) may include distal extension (242) which may stabilize pump (222) in the inferior vena cava, or at a junction between the inferior vena cava and the renal vein. For example, distal extension (242) may be a pigtail or j-shape. Pump (222) may also include anchoring mechanism (240), positioned on the cannula between inlet (236) and housing (226) through which blood exits the pump. Anchoring mechanism (240) may both anchor the pump (222) at a desired position along the inferior vena cava and partially occlude the inferior vena cava to allow operation of pump (222) across the anchoring mechanism.

In some embodiments, the anchoring mechanism may act as a valve to separate the inlet and outlet, such as to reduce recirculation and improve pump hydrodynamic performance characteristics. For example, anchoring mechanism (240) may anchor pump (222) between about 1-5 centimeters downstream of the renal vein. In another example, anchoring mechanism (240) may anchors pump (222) between about 2-3 centimeters downstream of the renal vein. In one example, anchoring mechanism (240) may be a balloon, which can be selectively inflated to at least partially occlude the inferior vena cava. Again, in this example, the anchoring mechanism may act to separate the inlet and outlet as a valve. For example, the size, shape, material, and position of the balloon on the cannula (220) may be selected to achieve different levels of occlusion in the inferior vena cava.

In another example, anchoring mechanism (240) may be an expandable cage. For example, anchoring mechanism (240) may be a self-expanding cage (e.g., Nitinol) which may be surrounded by a sheath for insertion and self-expands once the sheath is removed in situ. The cage may brace up against walls of the inferior vena cava and secure pump (222) in position. In one example, the cage may taper proximally and distally along the cannula, and be covered by a biocompatible cover material, to partially occlude the inferior vena cava.

Partial occlusion of the inferior vena cava, in combination with operation of pump (222), may draw blood from a location within the inferior vena cava and/or renal vein, to a location downstream of the pump inlet (236), may result in a pressure drop. The pressure drop may be measured as a pressure drop in the inferior vena cava upstream of pump (222) (e.g., proximate the renal vein), or a pressure drop in the renal vein. Alternatively, the pressure drop can be measured as a drop across the kidney between arterial pressure going into the kidney, and venous pressure coming out of the kidney (e.g., in the renal vein or transrenal pressure gradient). Indeed, in some aspects, the transrenal pressure gradient may be a parameter that is measured and used to guide therapy of the patient.

As with the first pump, pump (222) may also be percutaneously or surgically inserted into a patient, such as via the femoral vein or via the jugular vein or via the subclavian vein. In one example, pump (222) and pump (202) may be inserted through different percutaneous access points. Alternatively, pump (222) and pump (202) may be inserted through a same percutaneous access point (e.g., subclavian vein).

In one example, each pump (e.g., pump (202), pump (222)) may include one or more sensors, such as, e.g., a pressure sensor or a diagnostic sensor (e.g., a real time diagnostic sensor). For example, both pumps may include an integrated pressure sensor, such as a differential pressure sensor, a piezoelectric pressure sensor, or an optical pressure sensor. In FIG. 2B, a real-time diagnostic sensor (225) may be integrated into the second pump (222). In another example, a separate sensor may be provided, such as one introduced on a sensor wire, or a Swan-Ganz catheter.

In some examples, the diagnostic sensor (e.g., the real-time diagnostic sensor) may generate an electrical and/or optical signal proportional to a detected concentration of a compound filtered out of the blood by the kidneys, and the electrical signal may be generated sent to a controller (e.g., an Automated Impella Controller® (AIC) control device from Abiomed, Inc.). The signal may be displayed, or the signal may be converted (e.g., to a qualitative or quantitative value representative of the signal), and the converted values are displayed. For example, in some embodiments, multiple electrical and/or or optical signals may be gathered by the controller (e.g., multiple data points from different points in time), and a rate of change may be displayed.

Each pump (e.g., pump (202) and pump (222)) may be connected to a controller, e.g., an AIC, which receives data from the pump and the sensor associated with the pump (e.g., either an integrated sensor or a separate sensor), and generates for display to the user (e.g., a medical professional) information on cardiac output, and/or renal function (e.g., GFR, renal blood flow, urine output, natriuresis, etc.). As will be appreciated, in some embodiments, the pump may be connected to the same controller, although each pump may be connected to a separate controller. In such an example, each controller may be connected to an auxiliary monitor which may monitor and/or control the first and second controllers.

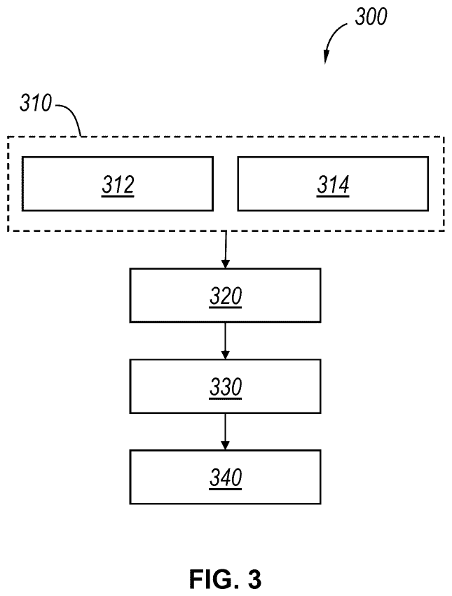

Referring to FIG. 3, the controller may be configured to receive (310) information. This may include receiving (312) data from a user (e.g., user-entered data). The user-entered data may include data related to renal function. As will be understood, data related to renal function will generally relate to date relevant to how the kidneys are functioning. This may include not just GFR, but urine output, natriuresis, protein urea, markers of renal dysfunction, etc.

Receiving information may include receiving (314) data related to renal function from a diagnostic sensor, such as the real-time diagnostic sensor.

In some embodiments, the data related to renal function may include one or more parameters related to renal function, as disclosed herein. In some embodiments, controller may be configured to derive or determine (320) the parameter(s) related to renal function based on the received information, including the data related to renal function. In some embodiments, a single parameter is determined or derived. In some embodiments, a plurality of different parameters is determined or derived.

The controller may be configured to adjust (330) a therapy delivered by the therapy-delivery device based on a parameter related to renal function. Adjusting the therapy may include increasing or decreasing therapy dose. Increasing or decreasing the therapy dose may include increasing or decreasing a rotational speed of a blood pump. Adjusting the therapy may include increasing or decreasing an intended duration of therapy. Adjusting the therapy may include increasing or decreasing a duty cycle of therapy. In some embodiments, adjusting the therapy may include replacing the blood pump with a blood pump capable of higher flow rates. In some embodiments, a first determined parameter may be used to adjust the therapy of the first pump while a second determined parameter may be used to adjust the therapy of the second pump. The first and second determined parameters may include measurements from the same or different areas of the patient (e.g., the venous system and/or the arterial system).

In some embodiments, the determined parameters may be compared to a prescribed value of renal function. In such embodiments, when the determined parameter falls outside of a prescribed value indicative of renal function, the controller may be configured to adjust the therapy being delivered to the patient. As will be appreciated, in such embodiments, the patients may have one or two installed mechanical support devices (e.g., pump (202) and/or pump (222)), with a set level of support. In such embodiments, in response to the determined value, adjusting the therapy may include increasing and/or decreasing the level of support to improve renal function.

In some embodiments, the controller may be configured to determine (340) adequacy of therapy withdrawal timing and/or duration of therapy based on the determined parameter related to renal function. For example, in some embodiments, after adjusting the therapy of the patient, the diagnostic sensor may sense and/or derive an additional parameter relating to kidney function (e.g., repeating some or all of step 310). In response to this value, the controller may be configured to determine if additional therapy is needed (e.g., at the same or different level) or if the support should be adjusted, such as if the sensed and/or derived parameter falls outside a prescribed value of renal function (e.g., outside an optimal range). In some embodiments, if the sensed and/or derived parameter is indicative of improved renal function (e.g., when compared to the desired value or range), the controller may be configured to suggest withdrawal and/or duration of the therapy.

As will be appreciated, steps 310-340 may continue to be repeated, such as every time the sensor is configured to sense one or more parameters of renal function.

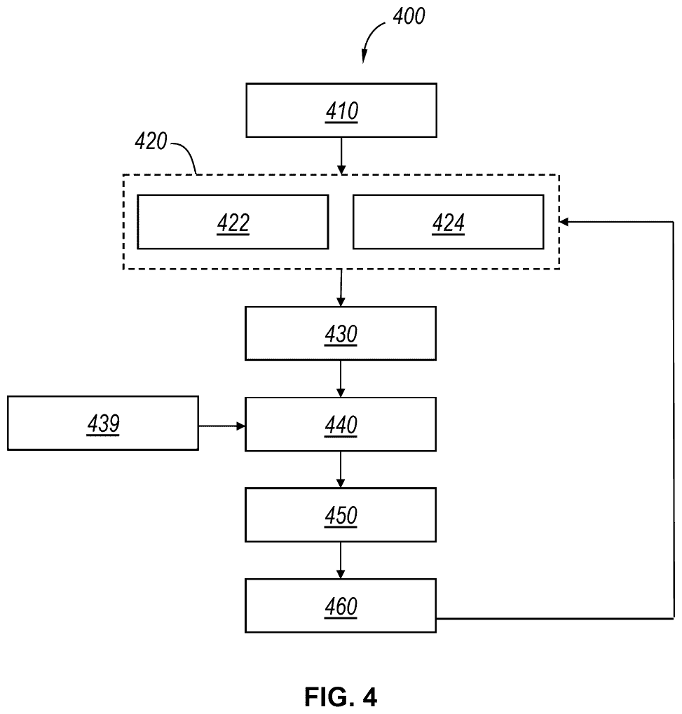

In various aspects, a method for cardiorenal syndrome (CRS) therapy may be provided. Referring to FIG. 4, the method (400) may include disposing (410) a therapy-delivery device within a patient. As disclosed herein, the therapy-delivery device may be mechanical circulator support device, such as a blood pump, a blood flow or pressure restrictor or enhancer, which may be disposed within a patient's arterial circulation or venous circulation. In some embodiments, the method may include inserting a single therapy-delivery device, while in other embodiments, the method may include inserting multiple therapy-delivery devices.

The method also may include receiving (420) information. In some embodiments, this may include receiving (422) data from a user (e.g., user-entered data). For example, the user-entered data may include data related to renal function. Receiving information also may include receiving (424) data related to renal function from a diagnostic sensor, such as a real-time diagnostic sensor. As described herein, the sensor may be pressure sensor (e.g., an integrated pressure sensor, such as a differential pressure sensor, a piezoelectric pressure sensor, or an optical pressure sensor). In some embodiments, the sensor may be integrated into one or both of the therapy-delivery devices. In other embodiments, a separate sensor may be provided, such as one introduced on a sensor wire, or a Swan-Ganz catheter.

In some embodiments, the data related to renal function may include one or more parameters related to renal function. In some embodiments, the method may include determining or deriving (430) parameter(s) related to renal function based on the received information, including the data related to renal function. In some embodiments, a single parameter is determined or derived. In some embodiments, a plurality of different parameters is determined or derived.

The method may include adjusting (440) a therapy delivered by the therapy-delivery device based on information related to renal function. In some embodiments, the method also may include starting therapy (439) delivered by the therapy-delivery device. In some embodiments, adjusting the therapy may include increasing or decreasing therapy, such as that selected for the patient when the therapy was started (e.g., in step 439). In embodiments in which therapy is not started, adjusting the therapy may include starting the therapy-delivery device. In some embodiments, increasing or decreasing the therapy may include increasing or decreasing a rotational speed of a blood pump. Adjusting the therapy may include increasing or decreasing an intended duration of therapy. Adjusting the therapy also may include increasing or decreasing a duty cycle of therapy.

The method may include determining (450) adequacy of therapy withdrawal timing and duration based on the parameter related to renal function, as described herein. The method may include causing (460) the therapy withdrawal timing and/or duration to be modified.

As will be appreciated, steps 420-460 may be repeated, such that the value of kidney function can be consistently monitored, with support adjusted accordingly.

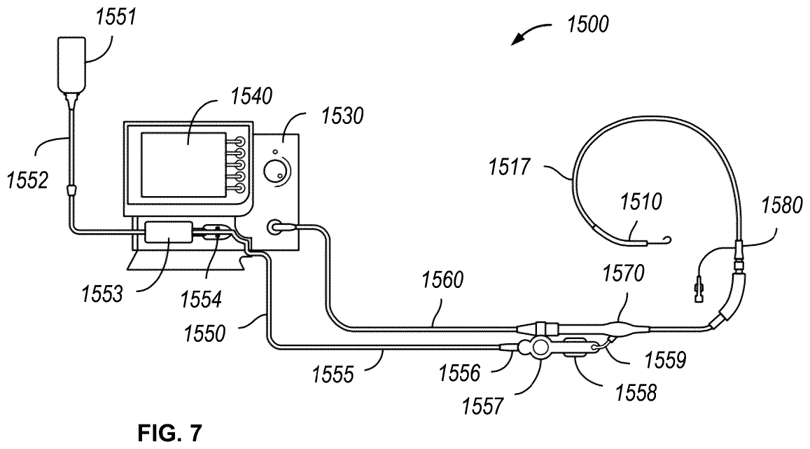

Referring to FIG. 7, a blood pump assembly (1500) may include a blood pump (1510) fluidically connected (e.g., through a tube (1552) to a container (1551) (such as a purge bag) that contains a purging fluid as disclosed herein, through a purging device (1553). The blood pump assembly (1500) also may include a controller (1530) (e.g., an AIC from Abiomed, Inc., Danvers, MA), a display (1540), a connector cable (1560), a plug (1570), and a repositioning unit (1580). As shown, the controller (1530) may include a display (1540). Controller (1530) may monitor and controls blood pump (1510). During operation, purging device (1553) may deliver a purge fluid (such as a saline-based purge fluid) to blood pump (1510) through a first line (1550, 1555) (e.g., a tube), through one or more components (1556, 1557, 1558, 1559) and through a catheter tube (1517), such as to prevent blood from entering the motor (not shown) within a motor housing of the pump. Connector cable (1560) may provide an electrical connection between blood pump (1510) and controller (1530). Plug (1570) connects catheter tube (1517), purging device (1553), and connector cable (1560). In some embodiments, plug (1570) may include a memory for storing operating parameters in case the patient needs to be transferred to another controller. Repositioning unit (1580) may be used to reposition blood pump (1510). As shown in this view, the fluid line (1550, 1555) may be separate from the connector cable (1560) having one or more electrical wires.

In another embodiment, GFR may be evaluated by measuring the value of a tracer introduced into the blood stream of a patient. For example, in some embodiments, a purge line of a percutaneous heart pump (e.g., a purge bag, such as container (1551) in FIG. 7, connected to a pump controller, such as controller (1530) in FIG. 7), may be spiked with a known concentration of a tracer (e.g., a fluorescent, autofluorescent, photoactivated, or other tracer). In some embodiments, the concentration and volume of the tracer may be provided to the controller. During use of the blood pump, the controller may be enabled to measure the provided value of the tracer to the patient. In such embodiment, the pump connected to the pump controller may include an optical sensor. In some embodiments, the optical sensor may be disposed on a portion of the pump, such as the cannula, that is blood contact (see, e.g., sensor (227) in FIG. 2C). For example, the sensor may be disposed on a tip of a cannula of the blood pump. In such embodiments, the sensor may be tuned to recognize the wavelength of the tracer, and the sensed data may be used to calculate the amount of tracer in the blood at any given time.

Using the sensed data, a difference between the amount of tracer provided to the patient and the amount of tracer removed the body may be calculated. In such embodiments, the difference may be compared to a prescribed “normal” range of the amount of tracer in the body. As will be appreciated, this range may vary depending upon the type of tracer selected, although the range also may be constant from tracer to tracer. In some embodiments, if the calculated difference is above the prescribed range (e.g., a residual concentration of tracer in the body is too high), a therapy describe herein may be prescribed to improve renal function. As will be appreciated, in some embodiments, the difference may be tracked over time to see if a concentration of tracer remaining in the body remains the same (e.g., too high) or decreases over time to be within, or even below the desired range.

In other embodiments, the residual concentration of tracer in body may be converted into a GFR value, indicating the speed of clearance. If the GFR falls outside the prescribed normal/healthy range as reported in literature, a renal therapy, such as those disclosed herein, may be performed. In such embodiments, the GFR may be tracked over time to see if the therapy allows the GRF of the patient to increase into (or even above) the prescribed range.

### EXAMPLES

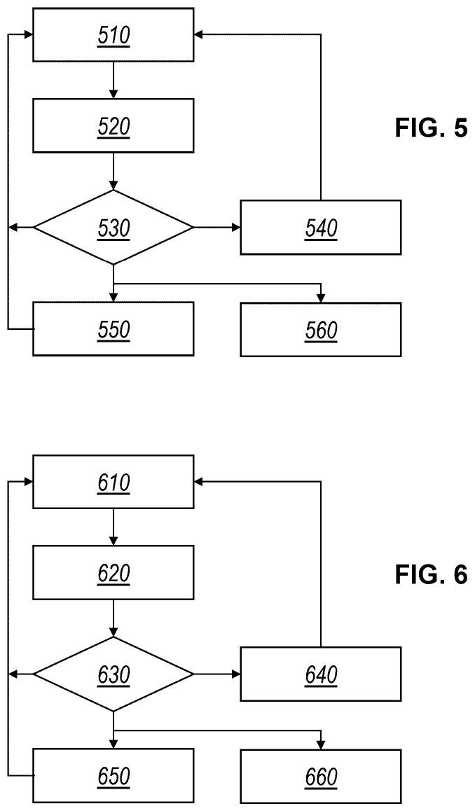

For example, referring to FIG. 5, in some embodiments, the controller may receive (510) data while a therapy-delivery device is in operation. A parameter (such as a GFR value) may be determined (520) based on sensor data. In some embodiments, a rate of change of the parameter may also be determined. The parameter and/or rate of change of the parameter may then be compared (530) to a threshold.

If the parameter is outside a predefined threshold, or is declining at a rate outside a threshold rate (which may be 0), the controller may be configured to increase (540) a pump's rotation speed until the parameter is within the threshold and/or the parameter is no longer declining.

If the parameter is increasing, or is stable, the controller may be configured to reduce (550) pump speed. Alternatively, the controller may indicate (560) (e.g., on a display) that the pump(s) may be removed.

As described herein, the steps may be repeated, as needed, as the patient's kidney function is monitored, and support is maintained and/or adjusted to improve renal function.

### GFR/eGFR

In some embodiments, a blood pump may be disposed in the arterial circulation to increase renal artery blood flow and pressure. In some embodiments, a blood pump may be disposed in the venous circulation to reduce renal vein pressure. In some embodiments, a first blood pump may be disposed in the arterial circulation, and a second blood pump may be disposed in the venous circulation.

The parameter used to control operation of the therapy-delivery devices may be a marker of renal function such as glomerular filtration rate (GFR). The parameter, or a rate of change of the parameter, may be determined based on sensor data.

The GFR can be estimated based on measured serum creatinine concentration corrected for patient demographics (estimated GFR, or eGFR). This method is the clinical standard.

However, eGFR it is inaccurate for some patients, is collected infrequently, and currently has a lengthy lag time from sample collection to results. In some instances, eGFR may lag actual AKI events by 24-48 hours.

The GFR can instead be directly calculated by measuring the clearance of a molecule from the plasma into the urine that is freely filtered by the kidney (e.g., inulin). This is a technically challenging method that is rarely conducted clinically, is collected infrequently, requires urine and plasma sampling, and has lag time (hours) from sample collection to results.

One measurement of GFR may be a direct measurement of GFR that is updated on a minute-by-minute basis and therefore allows for real-time adjustment of pump operation. One embodiment may use a fluorescently labeled tracer that is eliminated by the kidneys and optically detectable through the skin on a minute-by-minute basis. The change in this optical signal relates to the removal of the tracer via the kidneys and therefore GFR can be measured, and pump speed can be adjusted on a real-time basis.

### Urine Output/Diuresis

In some embodiments, a blood pump may be disposed, e.g., such as in the arterial circulation to increase renal blood flow and pressure. In some embodiments, a blood pump may be disposed, e.g., in the venous circulation to reduce renal vein pressure in order to increase diuresis. In some embodiments, a first pump may be disposed in the arterial circulation and a second pump may be disposed in the venous circulation.

The parameter used to control operation of the therapy-delivery devices may be a marker of diuresis such as urine output (UOP) or net fluid change (fluid input-UOP), or UOP relative to the amount of diuretic administered (i.e., UOP/diuretic dose or diuretic efficiency), weight change. The parameter, or a rate of change of the parameter, may be determined based on sensor data.

Urine output can be assessed by collecting urine volume over time or by using flow sensors on urine output collection tubing. Collection of urine volume over time is limited by the graduations of the collection vessel and often requires urine to be collected for 6 or more hours to collect large enough volumes to measure.

In some embodiments, UOP is measured using a flow sensor that measures flow of urine from the patient on a minute-by-minute basis and therefore allows for real-time adjustment of pump operation.

### Natriuresis

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to increase natriuresis. In some embodiments, a first pump may be disposed in the arterial circulation and a second pump may be disposed in the venous circulation.

The parameter used to control operation of the therapy-delivery devices may be a marker of natriuresis such as sodium concentration in the plasma or the urine or derivates thereof (e.g. fractional sodium excretion). The parameter, or a rate of change of the parameter, may be determined based on sensor data.

Urine and plasma sodium can be assessed by electrolyte biochemical assays conducted on the benchtop or using point of care tests. One method for utilizing plasma sodium concentration may include use of electrochemical sensors embedded on one or more of the blood contacting surfaces of the blood pumps. These electrochemical sensors could detect changes in electrical resistivity due to increasing amounts of the sodium electrolyte and thereby measuring sodium concentration on a minute-by-minute basis and therefore allows for real-time adjustment of pump operation.

### Hematocrit

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to facilitate hemoconcentration. In some embodiments, a first pump may be disposed in the arterial circulation and a second pump may be disposed in the venous circulation.

The parameter used to control operation of the therapy-delivery devices may be a marker of hemoconcentration such as hematocrit (i.e., the proportion of blood made up of blood cells). The parameter, or a rate of change of the parameter, may be determined based on sensor data.

One method of determining hematocrit may include use of optical sensors, which may be embedded on one or more of the blood contacting surfaces of the blood pumps. Due to the known effect of red blood cells on transmission/scattering of near infrared (NIR) wavelengths, a detected optical signal relating to the transmission/scattering of one or more NIR wavelengths may be used to estimate hematocrit on a minute-by-minute basis and therefore allows for real-time adjustment of pump operation.

### Decongestion

In some embodiments, a blood pump may be disposed, e.g., in the arterial circulation to increase renal blood flow and pressure, for example. In some embodiments, a blood pump may be disposed, e.g., in the venous circulation to reduce renal vein pressure in order to facilitate decongestion. In some embodiments, a first pump may be disposed in the arterial circulation and a second pump may be disposed in the venous circulation.

The parameter used to control operation of the therapy-delivery devices may be a marker of decongestion such as hemodynamic parameters (i.e., right atrial pressure, pulmonary capillary wedge pressure, pulmonary artery diastolic pressure, left ventricular end diastolic pressure), or the above parameters of UOP/diuresis, hemoconcentration, etc. The parameter, or a rate of change of the parameter, may be determined based on sensor data.

One method of determining decongestion may include use of pressure sensors, which may be embedded on one or more of the blood contacting surfaces of the blood pumps.

### Example 2

In some embodiments, a blood pump may be disposed, e.g., in the arterial circulation to increase renal blood flow and pressure. In some embodiments, a blood pump may be disposed, e.g., in the venous circulation to reduce renal vein pressure in order to provide nephroprotection from renal injury. In some embodiments, a first pump may be disposed in the arterial circulation and a second pump may be disposed in the venous circulation.

Referring to FIG. 6, in some embodiments, the controller may receive (610) data while a therapy-delivery device is in operation. A parameter may be determined (620) based on sensor data. In some embodiments, a rate of change of the parameter may also be determined. The parameter and/or rate of change of the parameter may then be compared (630) to a threshold.

If the parameter is outside a predefined threshold, or is increasing at a rate outside a threshold rate (which may be 0), the controller may be configured to increase (640) a pump's rotation speed until the parameter is within the threshold and/or the parameter is no longer increasing.

If the parameter is decreasing, or is stable, the controller may be configured to reduce (650) pump speed. Alternatively, the controller may indicate (660) (e.g., on a display) that the pump(s) may be removed.

### Renal Injury Biomarker

Here, the parameter used to control operation of the therapy-delivery devices may be a marker of renal injury such as NGAL, Cystatin C, TIMP-2, IGFBP-7, KIM 1, or a marker of renal oxygenation. The parameter, or a rate of change of the parameter, may be determined based on sensor data.

A preferred method of renal injury assessment could use standard laboratory values inputted into the pump control console where a predetermined algorithm adjusts pump speed. Another embodiment can embed the biochemical detection assay onto one or more of the blood contacting surfaces of the blood pumps and use optical or electrical changes related to the binding of the renal injury marker with a substrate to estimate renal injury on a minute-by-minute basis and therefore allows for real-time adjustment of pump operation.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

1. A system for cardiorenal syndrome (CRS) therapy, comprising:
a therapy-delivery device configured to be disposed within a patient, where the therapy-delivery device is a blood pump, a blood flow or pressure restrictor or enhancer;
a controller operably coupled to the therapy-delivery device, where the controller is configured to adjust a therapy delivered by the therapy-delivery device based on a parameter related to renal function.

2. The system of claim 1, wherein the therapy-delivery device is a blood pump disposed in a blood vessel forming part of a patient's arterial circulation.

3. The system of claim 1, wherein the therapy-delivery device is a blood pump disposed in a blood vessel forming part of a patient's venous circulation.

4. The system of claim 1, wherein the therapy-delivery device is a blood flow or pressure restrictor disposed in a blood vessel forming part of a patient's arterial circulation.

5. The system of claim 1, wherein the therapy-delivery device is a blood flow or pressure restrictor disposed in a blood vessel forming part of a patient's venous circulation.

6. The system of claim 1, wherein the therapy-delivery device is a blood flow or pressure enhancer disposed in a blood vessel forming part of a patient's arterial circulation.

7. The system of claim 1, wherein the therapy-delivery device is a blood flow or pressure enhancer disposed in a blood vessel forming part of a patient's venous circulation.

8. The system of claim 1, wherein the therapy-delivery device is a blood pump disposed in a blood vessel forming part of a patient's arterial circulation and a valve like structure is used to separate the inlet and outlet and improve pump hydrodynamic performance.

9. The system of claim 1, wherein the therapy-delivery device is a blood pump disposed in a blood vessel forming part of a patient's venous circulation and a valve like structure is used to separate the inlet and outlet and improve pump hydrodynamic performance.

10. The system of claim 1, wherein the parameter related to renal function is a hemodynamic parameter, a renal function parameter, or a combination thereof.

11. The system of claim 10, wherein the hemodynamic parameter is renal blood flow, cardiac index, or a combination thereof.

12. The system of claim 10, wherein the renal function parameter is serum creatinine concentration, urine creatinine concentration, blood urea nitrogen (BUN), urinary output, net fluid output (fluid input-urine output), serum sodium concentration, urinary sodium concentration, total urinary sodium content, blood concentration or hematocrit, change in body weight, estimated glomerular filtration rate (eGFR), direct glomerular filtration rate (GFR), a renal injury marker, or a combination thereof.

13. The system of claim 12, wherein the renal injury marker is Neutrophil gelatinase-associated lipocalin (NGAL), Cystatin C, TIMP-2, IGFBP-7, KIM 1, or a marker of renal oxygenation.

14. The system of claim 1, wherein the controller is configured to receive data from a user, the data including the parameter related to renal function.

15. The system of claim 1, wherein the controller is configured to receive the parameter related to renal function from a real-time diagnostic sensor.

16. The system of claim 15, wherein the real-time diagnostic sensor is integrated into the therapy-delivery device.

17. The system of claim 15, wherein the real-time diagnostic sensor is a separate device from the therapy-delivery device.

18-19. (canceled)

20. The system of claim 15, wherein the real-time diagnostic sensor is configured to detect an optical, electrical, and/or mechanical change in response to a measured biomarker.

21-26. (canceled)

27. The system of claim 1, wherein when the controller is further configured to determine adequacy of therapy withdrawal timing and duration.

28. A method for cardiorenal syndrome (CRS) therapy, comprising:
disposing a therapy-delivery device within a patient, where the therapy-delivery device is a blood pump, a blood flow or pressure restrictor or enhancer;
receiving information related to renal function;
adjusting a therapy delivered by the therapy-delivery device based on information related to renal function.

29-53. (canceled)
